(19) United States
(12) Patent Application Publication
(10) Pub. No.: US 2026/0256688 A1
(43) Pub. Date: Sep. 3, 2026

(54) INJECTABLE COMPOSITE DEPOT

(71) Applicant: DelSiTech Oy, Turku (FI)

(72) Inventors: Giancarlo MÉNDEZ AGREDA, Turku (FI); Mika JOKINEN, Turku (FI); Ari-Pekka FORSBACK, Turku (FI); Mika KOSKINEN, Turku (FI); Aarne MARTTILA, Turku (FI)

(21) Appl. No.: 19/489,951

(22) PCT Filed: Jun. 14, 2024
(86) PCT No.: PCT/FI2024/050316

§ 371 Date: Dec. 4, 2025

(30) Foreign Application Priority Data
Jun. 15, 2023 (FI) 20235676

(51) Int. Cl. A61K 9/00; A61K 9/50; A61K 31/192; A61K 31/198; A61K 47/12; A61K 47/26; A61K 47/36; A61K 47/69

(52) U.S. Cl. CPC A61K 9/0019; A61K 9/501; A61K 9/5089; A61K 31/192; A61K 31/198; A61K 47/12; A61K 47/26; A61K 47/36; A61K 47/6923

(57) ABSTRACT

The present invention relates to an injectable composite depot, which is shear-thinning. The composite depot comprises up to 85 weight-% of silica microparticles having a maximum diameter of ≤1000 μm, and preferably at least one encapsulated biologically active agent. The microparticles are combined with a silica sol having a silica content of ≤5 weight-%, preferably ≤2 weight-%, more preferably ≤1 weight-%. The injectable composite depot further comprises at least one pharmaceutically non-active additive encapsulated in the silica microparticles and/or present in the silica sol.

### BACKGROUND OF THE INVENTION

Minimally invasive injectable delivery systems utilizing thin syringe needles, e.g., suspensions, gels and gel-particle combinations, are attractive options for controlled drug delivery, especially for parenteral long-acting release of active pharmaceutical ingredients (APIs). Injectable delivery systems may also be utilized in topical administration, where a gel or a gel-particle combination can be utilized for sustained release of an API, e.g., in eye drops. Injectable formulations typically comprise both liquid and solid phases, for example gels, gel-particle combinations, and suspensions.

When dealing with different types of APIs, such as small-molecule drugs, peptides, proteins, polypeptides, fusion proteins, mRNA, vaccine antigens, viral vectors, adjuvants, lipid nanoparticles, and many more, there is sometimes a need to modify the components used in the injectable formulations to achieve the desired rheology and injectability as well as the desired controlled release rate. The presence of API may change the properties of both the solid and liquid phase of the injectable formulation, and the API may affect the solid and liquid phases in a different manner. Presence of the API in different concentrations both in the solid and liquid phase may further affect the release rate, and if the API is homogeneously distributed in the solid phase, e.g., in microparticles in suspensions or gels, also the surface chemistry of the solid phase may change from API to API, which affects the stability, rheology and the injectability of the suspension or the gel. The potency of APIs may vary a lot, and hence also the concentration of APIs in the injectables, also on the surface of particles, varies a lot.

Sometimes there is a need to fine-tune of the desired properties, such as controlled release rate and injectability, of the biodegradable materials used in the injectable formulations. Especially, if the release of the API is controlled by the biodegradation rate of the solid component of the injectable formulation in the body fluids, e.g., by dissolution rate of the microparticles with encapsulated or embedded API. If the required dose of the API is high, and/or injectability requires rheological properties, which can only be reached with high concentration of the solid component, it may result in high concentration of solid matter, e.g., microparticles in a suspension or in a gel. If the dissolution rate of the solid component, such as microparticles, also mainly controls the release rate of the API, a high concentration of the solid component in an injected dose may strongly retard the release rate. Depending on the location in the body and the local flow rates of the body fluids, the dissolution of the solid component may locally result in dissolution product concentrations above the sink condition (free dissolution), which retard the dissolution rate, and hence also the release of the API. In an extreme case the solid component dissolution may also saturate the body fluids locally in tissue with respect to the dissolution product. In that case the release rate of the API is fully dependent on the flow rate of the body fluids, i.e., on how fast the body fluids will be changed locally due to the flow. Hence, a high concentration of the solid component in the injectable controlled release depot based on the dissolution rate may narrow the possibilities to reach different release rates for the APIs. High local concentrations of dissolution products of a solid component may also cause undesired tissue responses and increase tolerability risks for the injectable depot.

WO 2014/207304 by Jokinen et al. discloses shear-thinning combined hydrogel compositions formed from spray-dried silica microparticles with encapsulated agents and silica sols. The disclosed shear-thinning hydrogel compositions are all-silica systems, where other solid components than biologically active agents or active pharmaceutical ingredients only comprise silica. The hydrogel compositions prepared from silica microparticles and silica sols, i.e. all silica depots, work in most cases very well in the injections through thin needles. However, when the silica microparticle to silica sol weight-to-volume ratio is lower than 0.5 the hydrogel structure can be weak, and the material is flowing and/or material is not injectable through thin needles. Also, in some cases the use of smaller amount of silica-based solids would be desirable for fine-tuning the release rates of API. The shear-thinning hydrogel compositions also work very well with many types of active pharmaceutical ingredients, in many payloads. However, with some APIs, there are sometimes challenges with the formation of the non-flowing hydrogel structure and injectability. For example, the silica microparticle surface may turn to be more hydrophobic and/or surface charge may change, for example due to the varying nanoscale roughness on the spray-dried silica microparticles or due to the encapsulated API. This may disturb wetting and gel formation, and further also injection of the hydrogel composition through a thin needle.

There is an interest to further improve the injectable composite depots and make it possible to use them with a variety of different APIs and biologically active agents. Furthermore, there is an interest to further modify the rheology and injectability of the injectable composite depots.

### OBJECT AND SUMMARY OF THE INVENTION

An object of this invention is to minimise or possibly even eliminate the disadvantages existing in the prior art.

One object of the present invention is an injectable composite depot with modified rheology and injectability.

A further object of the present invention is to provide an injectable composite depot with improved controlled release of pharmaceutically active ingredients and/or biologically active agents.

Another object of the present invention is to provide use for the injectable composite depot with an encapsulated agent or agents.

A further object of the present invention is to provide injectable composite depot with an encapsulated agent or agents for medical use.

These objects are attained with the invention having the characteristics presented below in the characterising parts of the independent claims.

Some preferred embodiments of the invention are presented in the dependent claims.

The embodiments mentioned in this text relate, where applicable, to all aspects of the invention, even if this is not always separately mentioned.

A typical injectable composite depot, which is shear-thinning and comprises
a) up to 85 weight-%, preferably up to 80 weight-%, of silica microparticles having a maximum diameter of ≤1000 μm, and preferably comprising at least one encapsulated biologically active agent, preferably pharmaceutically active agent, combined with
b) a silica sol having a silica content of ≤5 weight-%, preferably ≤2 weight-%, more preferably ≤1 weight-%,
wherein the injectable composite depot comprises at least one pharmaceutically non-active additive encapsulated in the silica microparticles and/or present in the silica sol.

A typical injectable formulation according to the present invention comprises the injectable composite depot according to the invention, preferably comprising a biologically active agent, more preferably a pharmaceutically active agent.

A typical use of the injectable composite depot of the present invention is for administering a biologically active agent or agents, preferably an active pharmaceutical ingredient or ingredients.

A typical injectable composite depot according to the present invention is for use as medicament.

### BRIEF DESCRIPTION OF THE DRAWINGS

FIGS. 1-3 illustrate injection forces needed for the injection through a 25 G needle (inner diameter 0.25-0.26 mm) for different injectable composite depots with and without pharmaceutically non-active additives in the silica microparticles and/or in the silica sol.

FIGS. 4-6 illustrate cumulative in vitro dissolution rate of silica and release rate of the active pharmaceutical ingredient with and without pharmaceutically non-active additives in the silica microparticles and/or in the silica sol.

FIG. 7 illustrates the viscoelastic properties, i.e. storage (elastic) and loss (viscous) modulus, for the injectable composite depots comprising silica microparticles, which are mixed with the silica sol with and without a pharmaceutically non-active additive.

### DETAILED DESCRIPTION OF THE INVENTION

The gist of the present invention is to improve rheological properties and injectability of silica-based injectable composite depots by using pharmaceutically non-active additives as rheology modifiers. It was surprisingly found that incorporation of a pharmaceutically non-active additive to the silica microparticles and/or to the silica sol of the injectable composite depot improves the rheological properties of the depot as well as the compatibility of the biologically active agents, such as active pharmaceutical ingredients, to the injectable composite depot. Some biologically active agents and active pharmaceutical ingredients (API) encapsulated in the silica microparticles, which are the major component in the injectable composite depot, may disturb the formation of the injectable composite depot structure, and hence also change its rheological properties and injectability. Now it has been found that pharmaceutically non-active additives can be used to overcome these challenges. The pharmaceutically non-active additives are unexpectedly effective in controlling and modifying the rheological propertied of the silica-based injectable composite depots. The opportunity to change rheological properties of the injectable composite depot also affect its other properties, which widen the possibilities to fine-tune the properties the injectable composite depot in parenteral administration of biologically active agents and/or active pharmaceutical ingredients (APIs). The pharmaceutically non-active additives can also be used to decrease the concentration levels of the biodegradable silica locally in tissue, which can be utilised in fine-tuning the release rate of biologically active agents and/or APIs. The changed concentration of the silica in the injectable composite depot may also have positive effect on local tolerability of the composite depot.

### Terms

A depot should be understood to be a drug delivery system, which is suitable for use for a controlled release of pharmaceutically active agents, such as active pharmaceutical ingredients, and/or biologically active agents.

Gel should be understood in the present context to be a homogeneous mixture of at least one solid phase and at least one liquid phase, preferably one liquid phase, i.e., a colloidal dispersion, where solid phase forms the continuous phase and the liquid phase is homogeneously dispersed in the continuous phase. The gel is viscoelastic and the elastic properties dominate at rest, which is indicated by rheological measurements under small angle oscillatory shear. The elastic properties dominate and the gel is non-flowing, when the loss factor (the loss tangent), tan δ=(G″/G′), of the gel is less than 1. The combined effect of the storage modulus (elastic modulus) G′ and the loss modulus (viscous modulus) G″ can also be expressed in the form of complex modulus (complex shear modulus), G*=G′+iG″. In the context of the present invention, the gel denotes hydrogel (see below), where the continuous phase comprises silica as such, silica as partly hydrolysed and/or silica as fully hydrolysed. Silica as such, silica as partly hydrolysed and/or silica as fully hydrolysed is the major component of the continuous solid phase.

The hydrogel should be understood to be a gel, where the liquid phase is water or where the liquid phase is water-based. The liquid phase of the hydrogel comprises more than 50 weight-% of water, calculated from the total weight of the liquid phase. Preferably the liquid phase of the hydrogel comprises >80 weight-%, more preferably >90 weight-% and even more preferably >97 weight-% of water. The liquid phase can additionally comprise small amounts of other liquids, typically organic solvents, e.g., ethanol. Typically the concentration of such solvents, e.g. ethanol, is <10 weight-%, more preferably <3 weight-% and even more preferably <1 weight-%, calculated from the total weight of the liquid phase. In the context of this invention the injectable composite depot of the invention is considered a hydrogel since it fulfils the basic criteria of a hydrogel.

The sol should be understood to be a homogeneous mixture of at least one liquid phase and at least one solid phase, i.e., a colloidal dispersion, or a colloidal suspension, where the liquid phase is the continuous phase, and the solid phase(s) are homogenously dispersed in the said liquid phase. In opposition to gel, the silica sol has clear flow properties and the liquid phase is dominating, i.e. the loss factor, tan δ=(G″/G′), of the sol is more than 1. In a silica sol, the liquid phase comprises mainly water, and optionally small amounts of ethanol and residuals of silica precursors. The solid phase(s) in a silica sol comprise colloidal nanoparticles of silica, partly or fully hydrolysed silica, aggregates of said nanoparticles or any combinations thereof.

A suspension should be understood to be a mixture of at least one liquid phase, preferably one liquid phase, and at least one solid phase, i.e., a dispersion, where the liquid phase is the continuous phase and the solid phase(s) are homogeneously dispersed in the liquid phase. In the present context, a suspension encompasses diluted mixtures of silica sol and silica microparticles, which preferably comprise biologically active agent. Suspension also encompasses mixtures of a silica sol, silica microparticles and a pharmaceutically non-active additive, e.g. alginate. Suspensions have clear flow properties and the liquid phase is dominating, i.e. the loss factor, tan δ=(G″/G′), of the sol is more than 1. As the suspensions are not colloidal, it is typical that mixing, preferably constant mixing, of the suspension is needed to ensure that the suspension remains stable.

Gel point or Gelation shall be understood in the present context to mean the point when a silica sol or a suspension that is flowing turns to a non-flowing and viscoelastic gel where the elastic properties dominate, indicated by rheological measurements under small angle oscillatory shear that the storage (elastic) modulus, G′ is greater than the loss (viscous) modulus and the loss factor is less than 1. The viscoelastic properties are commonly measured with a rheometer (a measuring device for determination of the correlation between deformation, shear stress and time) by the oscillatory shear, where shear stresses are small (small angles of deformation). The measurements are conducted by ensuring an adequate signal for a specific measuring system, i.e., a strain sweep is commonly done at constant frequencies to find the proper signal and the linear viscoelastic region for the rheometer system and then the actual measurements are done at constant strain with varying frequency. The varying frequencies give varying elastic and viscous modulus and the measurement show whether the solid or liquid phase dominates. In the form of a silica sol, the liquid state dominates, but the system contains varying amounts of solid phase(s) and the system is still flowing. Before the gel point it is typical that a steep increase in dynamic viscosity and storage (elastic) modulus is observed, which continues to rise after the gel point as the structure is developing. In the context of the present invention, the injectable composite depot is obtained after the combination of the silica microparticles and the silica sol, preferably comprising a pharmaceutically non-active additive, has reached the gel point. This means that the injectable composite depot has undergone gelation.

Injectable composite depot in the present context thus refers to a hydrogel, comprising at least one pharmaceutically non-active additive and preferably one or more pharmaceutically and/or biologically active agent(s). In the hydrogel of the injectable composite depot, the solid phase comprises silica microparticles and the liquid phase comprises water, optionally ethanol and/or residuals of silica precursors. The silica microparticles comprise the optional but preferable pharmaceutically and/or biologically active agent(s). The pharmaceutically non-active additive, e.g., alginate and/or citrate, may be present in the solid phase and/or in the liquid phase. The injectable composite depot is non-flowing and structurally stable when stored at rest, e.g., as stored in a syringe, and has shear-thinning behaviour under shear, i.e., decrease of viscosity and easy injection through 18-31 G needle (outer/inner diameter from 1.27/0.84 mm to 0.261/0.133 mm). The structural stability is indicated by rheological measurements under small angle oscillatory shear. Before the injection, e.g., as stored in a syringe and/or in an aluminium foil at temperatures <37° C., e.g., at room temperature of 20-25° C., or at refrigerator temperature of 2-8° C., the injectable composite depot is a gel, i.e., the storage (elastic) modulus G′ is greater than the loss (viscous) modulus G″ and the loss factor, tan δ=(G″/G′), is less than 1. When the storage (elastic) modulus is greater than the loss (viscous) modulus and the loss factor is less than 1, the injectable composite depot is non-flowing. Silica nanoparticles and/or aggregated silica nanoparticles of the silica sol merge to or integrate on the surface of the silica microparticles, wherein the injectable composite depot obtains its non-flowing and structurally stable structure, at the rest. Presence of silica nanoparticles is thus substantial for obtaining injectable composite depot. The non-flowing structure ensures the structural stability of the injectable composite depot by preventing the phase separation of the solid phase. In other words, the silica microparticles as well as the pharmaceutically non-active additive of the injectable composite depot are embedded in the hydrogel structure and they do not, e.g., precipitate or separate on the bottom of a vessel, e.g., a syringe, where the injectable composite depot is stored, typically at temperatures ≤25° C. Although the injectable composite depot is a hydrogel structure and remains stable and non-flowing as stored at rest, e.g., in a prefilled, ready-to-use syringe, the hydrogel structure of the injectable composite depot is so loose that it is shear-thinning when a shear stress, e.g., in the form of injection through a thin needle from a syringe is applied, e.g., by using 18-31 G needle (outer/inner diameter from 1.27/0.84 mm to 0.261/0.133 mm). The same rheological properties and injectability can also be utilised in topical administration of gel eye drops from squeezable devices, e.g., bottles, packs, and strips.

Injectable means, in the present context, parenteral administration via a surgical administration apparatus, e.g., a needle, a catheter or a combination of these, or topical administration of gel eye drops from squeezable devices, e.g., bottles, packs, and strips.

Shear-thinning in the present context denotes a rheological property of injectable composite depot. Whenever a shear rate of such an injectable composite depot is altered or shear stress is applied on the depot, the injectable composite depot will gradually move towards a new equilibrium state. At lower share rates the shear thinning composition is more viscous, and at higher shear rates it is less viscous. Thus shear-thinning refers to an effect where the viscosity of the injectable composite depot, i.e., the resistance to flow, decreases with an increasing rate of shear stress.

Biologically active agent in the context of this invention refers to any organic or inorganic agent that is biologically active, i.e., it induces a statistically significant biological response in a living tissue, organ, or organism. The biologically active agent can be a medicine, medicinal product, active pharmaceutical ingredient, vitamin, phytochemical, peptide, protein, fusion protein, polysaccharide, or a polynucleotide, e.g., DNA and RNA, vaccine antigen or viral vector. Preferably the biologically active agent is an active pharmaceutical ingredient.

In the context of this invention the term active pharmaceutical ingredient, API, refers to any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body. In the present context the terms “active pharmaceutical ingredient” and “pharmaceutically active agent” are used synonymously and they are wholly interchangeable with each other.

Encapsulated agents should be understood to be drugs, active pharmaceutical ingredients (APIs) or other therapeutically and/or biologically active agents, or delivery device, which is/are encapsulated into the silica microparticles of the injectable composite depot.

Pharmaceutically non-active additives in the context of this invention refers to any functional, pharmaceutically non-active ingredient, which improves the processing or properties of the injectable composite depot, e.g., gel formation, parenteral or topical administration or fine-adjustment of in vivo release rate of active pharmaceutical ingredients (API) and other therapeutic and biologically active agents. The pharmaceutically non-active additive is non-silica based or non-silica containing substance, compound or ingredient. The non-pharmaceutically active additive may be present in the silica sol and/or in the silica microparticles, i.e. encapsulated into the silica microparticles of the injectable composite depot.

In the present context solid content refers to the proportion of non-volatile material contained after the volatile solvent(s) has vaporized. More particularly it can refer to the solid content of the silica sol, or the solid content of a mixture of silica sol, pharmaceutically non-active additive, e.g., alginate, citrates, and optional other additives, or solid content of the silica microparticles, or solid content of the injectable composite depot comprising at least silica microparticles, silica sol, at least one pharmaceutically active additive and optional biologically active agent.

The all-silica depot, all-silica injectable depot, injectable silica depot or all-silica system refers to an injectable depot, where additives, such as alginate or citrates, have not been used.

### Features of the Invention

According to the present invention the injectable composite depot comprises at least one pharmaceutically non-active additive present, i.e. encapsulated, in the silica microparticles and/or present in the silica sol. The at least one pharmaceutically non-active additive functions as a rheology modifier. A pharmaceutically non-active additive, which affect the surface properties, can be incorporated in the microparticles and/or in the silica sol. When the pharmaceutically non-active additive is incorporated in the microparticles, it influences the rheological properties indirectly, through the changes in surface properties of the microparticles. Alternatively, or in addition, a pharmaceutically non-active additive, which directly change the rheological properties of the injectable composite depot, can be incorporated in the silica sol. The pharmaceutically non-active additive can be used to achieve desired target product profile for the injectable composite depot. Suitable pharmaceutically non-active additives can be added in the liquid phase and/or the solid phase during manufacture of the injectable composite depot in order to optimize the total amount of encapsulated agents, and/or the surface chemistry of the microparticles to obtain the desired rheological properties for the injectable composite depot. The pharmaceutically non-active additives may be useful also for the manufacture of injectable composite depots without any encapsulated agents or embedded APIs. Such injectable composite depots can be used as placebo materials, e.g., in preclinical in vivo experiments or in clinical experiments, or they can be added to increase the total solid content of the injectable composite depot for desired rheological properties.

The pharmaceutically non-active additives can be used affect, e.g. lower, the total solid content of the injectable composite depot. This is advantageous when the desired amount of biologically active agent, e.g. API, for a dose with a single shot can be reached already with a low concentration of microparticles in the injectable composite depot. In these cases, sometimes the microparticle concentration is not high enough to provide proper rheological properties for the injectability. This problem can now be solved by using pharmaceutically non-active additive, which can provide lower total solid content for the injectable composite depot with maintained rheological properties and injectability. When the concentration of the silica microparticles in injectable composite depot is lower, the dissolution products also have less effect on the release rate of biologically active agent, e.g. API, and there are more options for different controlled release rates in different locations in the body. The achieved impact is greater for injectable composite depots which are intended for use in locations with a small space and low body fluid flows, e.g., in the vitreous after intravitreal injections, but it also affects other common administration routes for the injectables, i.e., injections to subcutaneous and intramuscular tissue. The possible tolerability risks are also lower.

The pharmaceutically non-active additive may be a first additive selected from monosaccharides, disaccharides, oligosaccharides, polysaccharides, or their salts, or any combinations thereof; preferably from oligosaccharides, polysaccharides, or their salts or any combinations thereof; more preferably from polysaccharides or their salts, even more preferably from alginic acid, alginate or their salts, or from hyaluronic acid, hyaluronate or their salts, most preferably from alginic acid, alginate or their salts. Suitable monosaccharides may be selected, for example, from glucose, fructose, dextrose, galactose, or their mixtures; suitable disaccharides may be selected from sucrose, trehalose, maltose, lactose, or their mixtures, preferably trehalose; suitable oligosaccharides may be selected from melezitose, raffinose, or different broken down forms of polysaccharides, or any of their mixtures. Suitable polysaccharides may be selected from starch, glycogen, chitin, chitosan, dextran with different molecular weights, cellulose, cellulose derivatives, or their mixtures. The first additives, i.e. monosaccharides, disaccharides, oligosaccharides and polysaccharides, are able to form gels and shear-thinning structures with water. Since they directly affect rheological properties of the injectable composite depot, the first additives are preferably incorporated in the silica sol of the injectable composite depot for maximal effect. Incorporation of a first additive into the silica microparticles would not be expected to improve gel properties of the final injectable depot in a similar degree. According to one preferable embodiment of the present invention, the first additive, present in the silica sol, is selected from alginic acid, alginate or their salts. Preferably the first additive, present in the silica sol, is sodium alginate.

The first additive selected from monosaccharides, disaccharides, oligosaccharides, polysaccharides, their salts or any combinations thereof, when incorporated in the silica sol, it is able to form gels and/or shear-thinning structures in absence of crosslinkers, such as crosslinking salts, for example calcium chloride. According to the one preferable embodiment, calcium salts of alginic acid are excluded from the possible first additives. Thus the first additive is not calcium alginate, i.e. the injectable composite depot is free of calcium alginate.

According to one embodiment the pharmaceutically non-active additive may be present in the silica sol for affecting the rheological properties of the injectable composite depot. Preferably the silica sol comprises a first additive selected from monosaccharides, disaccharides, oligosaccharides, polysaccharides, or their salts, or any combinations thereof. According to one specific embodiment, the silica sol comprises the first additive which is trehalose. It was unexpected that the first additive selected from monosaccharides, disaccharides, oligosaccharides, polysaccharides, or their salts, was able to function as rheology modifier even when the molecular size of the first additive was relatively small, e.g. monosaccharide or disaccharide, such as trehalose.

It is assumed, without wishing to be bound by any theory, that the function of the first additive selected from monosaccharides, disaccharides, oligosaccharides, polysaccharides or their salts, such as alginic acid, alginate or their salts, hyaluronic acid, hyaluronate or their salts, or trehalose, is based on their said ability to form gels or high viscous fluids. Together the silica microparticles, silica sol comprising silica nanoparticles and/or aggregates of silica nanoparticles and the first additive are able form an improved injectable composite depot, preferably in the absence of crosslinking salts or the like. In general, the silica nanoparticles contribute to the gel formation by merging onto the surface of silica microparticles. The desired rheological properties, i.e. to have simultaneously a non-flowing material at rest (as in a prefilled syringe in storage) and appropriate shear-thinning viscosity under stress (e.g., when injecting the depot through a thin needle), can be obtained when silica microparticles, silica sol with the silica nanoparticles and appropriate concentrations of the first additive are combined. It is thus possible to tailor the properties of the injectable composite depot by a selecting appropriate combination of silica microparticles, silica nanoparticles in the silica sol, and the first additive, such as alginate. This can be done by a person skilled in the art by using a limited number of experiments.

The presence of the first additive in the silica sol affects the hydrogel formation mechanism and provides a possibility to control the final rheological properties of the injectable composite depot. The presence of the first additive selected from monosaccharides, disaccharides, oligosaccharides, polysaccharides or their salts in the silica sol may thus be used to adjust and/or fine-tune the rheological properties of the injectable composite depot. The first additive may function as an effective rheology modifier, if needed. With the first additive it is possible to balance or counteract rheology changes caused by the active pharmaceutical ingredient present in the silica microparticles. For example, the first additive, e.g. alginic acid, alginate or trehalose, may be used to adjust the viscosity and viscoelastic properties of the injectable composite depot (e.g. decrease the viscosity and storage (elastic) modulus or increase the viscosity and storage (elastic) modulus), if the active pharmaceutical ingredient in the silica microparticle has changed the surface chemistry of the silica microparticles and correspondingly the rheology of the depot in a non-desired manner.

The first additive, such as monosaccharides, disaccharides, oligosaccharides, polysaccharides, their salts, or any combinations thereof, in the silica sol may have a concentration in a range of 0.25-1.5 weight-%, preferably 0.50-1.25 weight-%, more preferably 0.75-1.25 weight-% or 0.75-1.0 weight-%, calculated from the total weight of the silica sol and the pharmaceutically non-active additive(s) present in the sol. The first additive (especially monosaccharides, disaccharides, oligosaccharides, polysaccharides, alginic acid, alginate, hyaluronic acid, trehalose) has been observed to be compatible with the silica microparticles and silica nanoparticles in the silica sol to provide non-flowing gel structure at rest, fast gel formation, and/or provide easily injectable composite depots. These improvements can be achieved especially when the microparticle concentration in injectable composite depot is low (e.g., microparticle to silica sol weight-to-volume ratio 0.5:1.0 or lower). By using 0.25-1.5 weight-%, preferably 0.50-1.25 weight-%, more preferably 0.75-1.25 weight-% or 0.75-1.0 weight-% of the first additive in the silica sol, the silica microparticle to silica sol weight-to-volume ratio can be in a range from 0.1:1.0 to 0.8:1.0, from 0.2:1.0 to 0.7:1.0, from 0.3:1.0 to 0.6:1.0, from 0.4:1.0 to 0.5:1.0. According to another embodiment, the silica microparticle to silica sol weight-to-volume ratio can be in a range from 0.1:1.0 to 0.5:1.0, from 0.2:1.0 to 0.4:1.0, from 0.3:1.0 to 0.4:1.0. In some embodiments even lower silica microparticle concentrations may be appropriate, depending on the specific properties of the API or the surface properties in general. The silica microparticle to silica sol weight-to-volume ratio may be from 0.08:1.0 to 0.9:1.0 or 0.09:1.0 to 0.5:1.0. For all weight-to-volume ratios given here, the silica sol includes the pharmaceutically non-active additive, if present in the silica sol. Even at these low silica microparticle concentrations, the use of the first additive provides the injectable composite depot with rheological properties that are at least comparable with conventional all-silica systems (with silica microparticle to silica sol weight-to-volume ratio 1.0:1.0). The rheological properties may be even better, indicated for example in lower force needed in injection through thin needles, such as 18-31 G with inner diameters between 0.13-0.84 mm, and/or less changes in the force needed during the injection.

When the use of pharmaceutically non-active additive enables the lower concentration of the silica microparticles in the injectable composite depot, the local tissue concentrations of dissolved silica will also be lower when injecting corresponding depot volumes (e.g., 50 μl-1 ml) as for the conventional all-silica depots. Based on in vitro dissolution studies, it has been observed that injectable composite depots with pharmaceutically non-active additive, especially alginate, have in vitro similar prolonged release properties for biologically active agents, e.g. API, and silica, compared to corresponding silica microparticles of conventional all-silica systems. This is also indicating that the injectable composite depots of the present invention comprising pharmaceutically non-active additive can be used to produce similar prolonged release drug products that can be produced with the conventional all-silica systems. In addition, pharmaceutically non-active additive containing injectable composite depots can also be used in situations where conventional all-silica depots do not work, at least optimally.

According to one embodiment of the invention, the pharmaceutically non-active additive may comprise a second additive selected from citric acid or salts of citric acid, preferably from monosodium citrate or its hydrates, disodium citrate or its hydrates, or trisodium citrate or its hydrates, and most preferably from trisodium citrate or its hydrates. Use of citric acid or its salts in the injectable composite depot has been observed to improve the injectability of the composite depot especially in cases where encapsulated agents in the silica microparticles, such as active pharmaceutical ingredients or other biologically active agents, have changed the surface properties of the silica microparticles in a manner which worsens the injectability of the injectable composite depot. Often the changes in the surface properties are observed in 1) poor wetting of the silica microparticles in the silica sol, 2) prolonged gel formation, and/or 3) in formation of a weak gel. It is speculated that the reason for changes may be fewer contact points for silica nanoparticles to merge on the silica microparticle surface, different surface chemistry when certain API molecules are located at the microparticle surface (when API is homogeneously distributed in a silica microparticle, some API molecules will be near or at the surface of the silica microparticle) and/or changed surface charge. Without using pharmaceutically non-active additive according to the present invention, in some cases the obtained weak gels might be a subject to partial phase-separation due to a high shear stress under the injection through a thin needle. If phase separation occurs, more force may be needed in the injection or in the worst case the needle may even get clogged. Already a high force used in the injections, e.g., in delicate administrations, such as in intravitreal or subconjunctival injections, may cause risk of adverse effects due to injection. These disadvantages can be eliminated or at least alleviated when a second additive is present in the injectable composite depot.

The second additive may have a concentration ≤5 weight-%, preferably ≤3 weight-%, more preferably ≤2 weight-%. The concentration may preferably be in range of 0.1-5 weight-%, preferably 0.1-3 weight-%, sometimes 0.2-2 weight-%, calculated from a total weight of the silica in the injectable composite depot. It has been observed that these concentrations of the second additive improve the injectability of the composite depot, for example when levothyroxine is used as active pharmaceutical ingredient.

The second additive may be encapsulated or present in the silica microparticles and/or present in the silica sol. This means that citric acid or its salts, such as citrate, can be incorporated either into the silica microparticles during the manufacture of the silica microparticles and/or added to the silica sol during the manufacture of injectable composite depot. In both cases the second additive, i.e. citric acid or its salts, affects the surface properties of the silica microparticles and/or the silica nanoparticles which are part of the silica sol, either by being directly part of the silica microparticle or through ionic interactions. The second additive works also for particles that have a silica surface, e.g., solid particles of active pharmaceutical ingredients that have been coated with a silica surface.

The second additive can be incorporated or present as citric acid or its salt, such as monosodium citrate, disodium citrate or more preferably as trisodium citrate. Salts of citric acid, citrates, can be used in anhydrous form or as water containing form, such as trisodium citrate dihydrate.

According to one embodiment of the invention the pharmaceutically non-active additive(s) present or encapsulated in the silica microparticles and the pharmaceutically non-active additive(s) present in the silica sol may be different from each other. For example, the injectable composite depot may comprise a first additive present in the silica sol and a second additive encapsulated in the silica microparticles. Use of at least two or more pharmaceutically non-active additives in the injectable composite depot provides increased versality and possibility to use various biologically active agents, such as APIs. Pharmaceutically non-active additives that can affect surface charges of the nanoparticles and/or microparticles, such as citric acid and its salts, can be incorporated both to the silica microparticles and into the silica sol. This makes it possible to affect surface properties more than they would be affected when the pharmaceutically non-active additives would be only in microparticles or only in silica sol. Additives that directly affect the rheological properties of the injectable composite depot, such as mono-, di-, oligo- and polysaccharides, such as alginate, can be incorporated to the silica sol while another additive which affects surface properties, such as citric acid or its salt, is incorporated into the silica microparticles. According to one preferable embodiment, combination of citrate in the silica microparticles and alginate in the silica sol in injectable composite depot has been shown to provide excellent properties.

The pharmaceutically non-active additives can be incorporated to the silica microparticles during the manufacture of silica microparticles and/or to the silica sol during the manufacture of injectable composite depot.

The injectable composite depot may be obtained by combining the silica microparticles with silica sol, for example by mixing.

The injectable composite depot comprises up to 85 weight-%, preferably up to 80 weight-%, of silica microparticles having a maximum diameter of ≤1000 μm. According to one preferable embodiment the injectable composite depot may comprise 5-85 weight-%, preferably 5-80 weight-%, more preferably 5-50 weight-%, of the silica microparticles, calculated from the total weight of the injectable composite depot. According to another embodiment of the injectable composite depot may comprise 5-50 weight-%, preferably 5-30 weight-%, sometimes 5-25 weight-%; of the silica microparticles, calculated from the total weight of the injectable composite depot.

The silica microparticles of the injectable composite depot can be selected from spray-dried silica microparticles, silica fibre fragments, moulded or casted silica monoliths as such and/or crushed, or any of their mixtures. Preferably silica microparticles are obtained by sol-gel process and spray-dried.

It is possible that the silica content in the silica microparticles can be in a range of 20-99.9 weight-%, preferably 30-99.9 weight-%, more preferably 50-99.7 weight-%, more preferably 70-99 weight-%, calculated from the total weight of the silica microparticles. The silica content of the silica microparticles may be at least 20 weight-%, preferably at least 30 weight-%, more preferably at least 40 weight-%, sometimes at least 50 weight-%, calculated from the total weight of the silica microparticles. The silica content can be in a range of 30-95 weight-%, preferably 40-95 weight-%, more preferably 50-93 weight-% or 70-90 weight-%, calculated from the total weight of the silica microparticles. In addition to silica, the silica microparticles may comprise pharmaceutically non-active additives and pharmaceutically active agents and/or biologically active agents.

The silica microparticles of the injectable composite depot have a maximum diameter of ≤1000 μm. According to one embodiment, the injectable composite depot comprises silica microparticles, which may have a diameter in a range of 1-300 um, preferably 1-100 μm, more preferably 1-30 μm, even more preferably 0.5-20 μm, sometimes even 0.5-15 μm. Particle size can be measured by using laser diffraction. The use of pharmaceutically non-active additive increases the degree of freedom in selection of microparticle diameter, as it is possible to use even silica microparticles with small diameter without deterioration of the rheology of the injectable composite depot.

The injectable composite depot further comprises a silica sol having a silica content of ≤5 weight-%, preferably ≤2 weight-%, more preferably ≤1 weight-%. The silica content may be in a range of 0.1-5 weight-%, preferably 0.5-2 weight-%, more preferably 0.6-1 weight-%, calculated from the total weight of the silica sol. The silica sol comprises silica nanoparticles as a solid phase and water, and optionally even a small amount of alcohol, such as ethanol, as a liquid phase. The silica sol comprises more than 50 weight-% of water, calculated from the total weight of the silica sol. Preferably the liquid phase of the silica sol comprises >80 weight-%, more preferably >90 weight-% and even more preferably >97 weight-% of water. The liquid phase of the silica sol can additionally comprise other liquids, typically organic solvents, e.g., ethanol. Typically, the concentration of such solvents, e.g. ethanol, is <10 weight-%, preferably <3 weight-%, more preferably <1 weight-%, even more preferably <0.5 weight-%, calculated from the total weight of the silica sol.

According to one embodiment of the invention, the injectable composite depot comprises silica sol which comprises silica nanoparticles having a diameter in a range of 10-1000 nm, preferably 10-500 nm, and more preferably 10-190 nm. The particle diameter can be determined by using dynamic light scattering. The pharmaceutically non-active additives make it possible to use nanoparticles with a small diameter in the silica sol while maintaining the properties of the injectable composite depot within appropriate limits. Silica nanoparticles in the silica sol contribute to the gel formation as they merge to the surface of much larger silica microparticles, wherein the whole structure turns into a non-flowing hydrogel structure, which is easily injectable through thin needles (e.g., 18-31 G with inner diameters between 0.13-0.84 mm), and again back into a non-flowing hydrogel after the injection in tissue. Together the silica microparticles and the silica nanoparticles thus ensure the development of a non-flowing gel structure (at rest), which also is easily injectable through thin needles, i.e., the gel structure is strongly shear thinning under shear.

The injectable composite depot preferably comprises at least 15 weight-%, more preferably at least 20 weight-%, even more preferably at least 50 weight-%, of silica sol. The amount of silica sol in the injectable composite depot can be, for example in a range of 15-95 weight-%, preferably 20-95 weight-%, more preferably 50-95 weight-%, of silica sol, calculated from the total weight of the injectable composite depot.

The total silica content in the injectable composite depot may be ≤85 weight-%.

According to one embodiment of the invention, the injectable composite depot may comprise 15-95 weight preferably 20-95 weight-%, more preferably 50-95 weight-% or 50-90 weight-%, of water, calculated from the total weight of the injectable composite depot.

The solid content of the injectable composite depot may be 5-85 weight-%, preferably 5-80 weight-%, more preferably 5-50 weight-%, sometimes 10-50 weight-%, calculated from the total weight of the injectable composite depot.

The pharmaceutically non-active additives can be useful in improving processing or properties of injectable composite depots comprising any type of biologically active agents, including therapeutic agents or active pharmaceutical ingredients. According to one preferable embodiment the silica microparticles comprise at least one encapsulated pharmaceutically active agent and/or biologically active agent. The pharmaceutically active agent and/or biologically active agent can be selected from small-molecule drugs, vitamins, phytochemical, peptides, proteins, fusion proteins, nucleic acids (e.g., DNA, RNA), vaccine antigens, viral vectors or liposomes carrying a biologically active agent.

The silica microparticles may comprise, for example, 0.01-70 weight-% or 0.1-70 weight-%, preferably 0.3-50 weight-%, more preferably 1-30 weight-%, of pharmaceutically active agent(s) and/or biologically active agent(s), calculated from the total weight of silica microparticle.

The pharmaceutically non-active additives can be useful in improving processing or properties of injectable composite depots comprising any type of biologically active agents, including therapeutic agents or active pharmaceutical ingredients, such as small-molecule drugs, vitamins, phytochemical, peptides, proteins, fusion proteins, nucleic acids (e.g., DNA, RNA), vaccine antigens, viral vectors or liposomes carrying a biologically active agent.

The present invention is especially suitable for biologically active agents, such as pharmaceutically active agents, having several pKa values, high charge and/or amphoteric properties. For example, peptides, polypeptides and proteins may have different charges in the same pH, and the pKa ranges vary, fusion proteins may have a complex charge structure, high negative charge of nucleid acids may also in some cases be challenging for providing injectable composite depots. The use of pharmaceutically non-active additives improves the gel formation, injectability, and/or fine-adjustment of in vivo release rates. Even some vitamins, e.g., B12 and C vitamins, seem to benefit from the use of the pharmaceutically non-active additives, which may be used to improve the properties, such as injectability, of the injectable composite depot.

According to one embodiment, the pharmaceutically active agent may be Levothyroxine. Levothyroxine with several pKa values at 2.2, 6.7, and 10.1 seems to affect the wetting of the silica microparticles, and consequently the gel formation and injectability. It has been observed that incorporation of pharmaceutically non-active additive, both in the silica microparticles and in the silica sol, significantly improved the properties of the injectable composite depot. For example, the pharmaceutically active agent can be Levothyroxine when citrate is encapsulated in the silica microparticles and alginate is present in the silica sol.

According to one embodiment, the biologically active agent is a vaccine antigen or viral vector, which are often large and complex structures with varying chemical structures, which may cause challenges in the fine-adjustment of the properties of the injectable composite depots comprising them. Hence, the pharmaceutically non-active additives, such as alginate and citrates, are good tools to improve the properties of the silica-based injectable depot platform technology, which is used to prepare controlled delivery systems for any type of biologically active agents and pharmaceutically active ingredients.

The injectable composite depot can be used for administering a biologically active agent or agents, preferably an active pharmaceutical ingredient or ingredients. The administration may be topical or parenteral.

According to one embodiment the administration may be topical, wherein a biologically active agent is administered either as eye drops, creams, gels, ointments, lotions, or suspensions.

According to one preferable embodiment the injectable composite depot is suitable for use as or in a topical ophthalmic formulation, such as eye drop, eye cream or eye gel. The injectable composite depot may be suitable for administration topically in form of eye drops, eye gel or eye cream. When the injectable composite depot is intended for ophthalmic formulations, the pharmaceutically non-active additive present in the silica sol is preferably selected from one or more of the first additives as listed above, especially trehalose. The injectable composite depot intended for ophthalmic formulations may further comprise the pharmaceutically non-active second additive selected from citric acid or salts of citric acid, preferably from monosodium citrate or its hydrates, disodium citrate or its hydrates, or trisodium citrate or its hydrates. The second additive may be present in ophthalmic formulations, such as eye drops, eye creams, eye gels, as encapsulated in the silica microparticles and/or present in the silica sol.

According to another embodiment, the administration may be parenteral and selected from the group consisting of intravenous, intraarterial, intracardiac, transdermal, transmucosal, intradermal, subcutaneous, intramuscular, intraperitoneal, intracerebral, intracerebroventricular, intrathecal, intraosseous, intraarticular, ophthalmic, intraocular, intravitreal, subconjunctival, intracameral, subretinal, retrobulbar, peribulbar, suprachoroidal, periocular, transscleral, intrasternal, posterior juxtascleral, sub-tenon, intravesical and intracavernosal.

### EXAMPLES

Some embodiments of the invention are illustrated in the following non-limiting examples. The examples show how pharmaceutically non-actives additives are used in different silica sols. Some of the silica sols are used to prepare silica microparticles by spray-drying, and some silica sols, i.e., very diluted silica sols, such as R400 silica sol (molar water-to-TEOS ratio is 400) are only used in the preparation of the final injectable composite depot. The active pharmaceutical ingredients are mainly encapsulated in the silica microparticles. Diluted silica sol (R400) provides the liquid phase and silica nanoparticles that are needed together with the spray-dried silica microparticles to prepare a non-flowing gel structure (at rest), which also is easily injectable through thin needles, i.e., the gel structure is strongly shear thinning under shear. The silica nanoparticles in the silica sol contribute to the gel formation as they merge to the surface of much larger silica microparticles. In some cases, the function of the silica sol can be enhanced or improved by incorporation of pharmaceutically non-active additive.

### Example 1—Improved injectability for Injectable Composite Depots using Pharmaceutically Non-Active Additives in Silica Microparticles

Silica microparticles with and without fluorescein, and the silica microparticles comprising both fluorescein and pharmaceutically non-active additive (trisodium citrate) were prepared.

A silica sol for spray-drying of silica microparticles was prepared by hydrolyzing tetraethyl orthosilicate (TEOS) in water, of which pH was adjusted to pH 2 using 0.1 M HCl. The molar water-to-TEOS ratio was 10. After the hydrolysis the sol was cooled down to ca. 0° C. in an ice-bath. The active model substance was fluorescein (free base, payload related to the silica amount was 5 weigh-%) and the pharmaceutically non-active additive to improve injectability was trisodium citrate dihydrate (0.3 weight % in relation to the silica amount in the sol). Fluorescein and trisodium citrate were dissolved in given concentrations into ethanol and the solution was combined with the silica sol prior to the spray-drying (added volume of ethanol corresponded the volume of water increasing the molar water-to-TEOS ratio to 100). The pH of the mixture was adjusted to ca. 6.3 with by using 0.1 M NaOH.

In the spray-drying of the silica microparticles with Buechi-191 spray-dryer, the inlet temperature was 120° C., outlet temperature 78° C., aspirator at 35 m³/h, pump at ca. 4 ml/min and the atomization air flow was at 700 l/h. The silica microparticles without fluorescein were prepared with the GEA Niro Mobile Minor spray-dryer, and the inlet temperature was 180° C., outlet temperature ca. 90° C., aspirator was at 80 kg/h, pump at 35 g/ml, and the atomization air flow was at 11.8 kg/h.

Injectable composite depots for each of the silica microparticle batches described above were prepared by mixing them into a silica sol (R400) at pH 6.2. The silica sol (R400) was made by hydrolyzing TEOS in molar water-to-TEOS ratio of 400 at pH 2, and after the hydrolysis, the pH was adjusted to pH 6.2. The silica microparticles were mixed with the silica sol in 1:1 ratio (weight-to-volume), and the resulting mixtures were transferred into syringes (BD, 1 ml, Luer-lok™ Tip), and the syringes were attached into a roller mixer, and they were kept at room temperature. The mixtures formed the injectable composite depots, a non-flowing hydrogel structure within 72 hours.

The injectable composite depots were then tested by manual injections through thin needles. All the injectable composite depots comprising pharmaceutically non-active additive (trisodium citrate), and the depot comprising silica microparticles without any encapsulated agents could be easily injected through a 25-27 G (inner diameter 0.21-0.26 mm) needle. The composite depots comprising fluorescein, but no pharmaceutically non-active additive (trisodium citrate) were more difficult to inject: they could be in the best cases injected with 18-20 G needles (inner diameters 0.337-0.838 mm). Differences in the particle size distributions (measured by using Sympatec HELOS 2370 laser diffraction instrument) were small. Particle sizes D10 and D50 were almost identical, but with the pharmaceutically non-active additive (trisodium citrate) particle size D90 was a bit lower, i.e., they do not explain the differences in the injectability. It can be concluded that the presence of pharmaceutically non-active additive (trisodium citrate) in the silica microparticles clearly improved the wetting, and the non-flowing gel structure formed much faster, which indicates changes in the surface structure.

Another molecule, Bromophenol blue, was also studied as a model molecule for an active substance with and without pharmaceutically non-active additive (trisodium citrate) in the silica microparticles.

For the silica microparticles with bromophenol blue as the only encapsulated agent (no pharmaceutically non-active additive present), a silica sol comprising 50 ml of tetraethyl orthosilicate (TEOS), 31.35 ml deionised water and 9.04 ml of 0.1 M HCl was made. The silica sol was then mixed until TEOS was hydrolysed. After the hydrolysis, the silica sol was cooled down to ca. 0° C., and it was diluted by adding 100 ml ethanol, and the pH was adjusted to pH 5.0 by adding 0.1 M NaOH. Next, 40 ml of a solution comprising 1.35 g bromophenol blue dissolved in water was added into the silica sol, and the mixture was pumped into a spray-dryer Buechi B-191, where inlet temperature was 120° C., aspirator at 95%, pump at 16% and the atomisation air flow at 600 ml/h.

Another silica sol was made for preparation of silica microparticles where both bromophenol blue and pharmaceutically non-active additive (trisodium citrate) were encapsulated in the silica microparticles. 50 ml of tetraethyl orthosilicate (TEOS), 31.35 ml deionised water and 9.04 ml of 0.1 M HCl was made. The silica sol was then mixed until TEOS was hydrolysed. After the hydrolysis, the silica sol was cooled down to 0° C., and it was diluted by adding 100 ml ethanol, and the pH was adjusted to pH 5.0 by adding 0.1 M NaOH. Next, 40 ml of a water solution comprising pharmaceutically non-active additive (trisodium citrate hydrate) at concentration of 57.2 mM (resulting in the payload of 4.33 w-% in relation to the silica amount) and 1.35 g bromophenol was added into the silica sol, and the mixture was pumped into a spray-dryer Buechi B-191, where inlet temperature was 120° C., aspirator at 95%, pump at 16% and the atomisation air flow at 600 ml/h.

The silica microparticles with and without trisodium citrate were used both as suspensions in water, and as injectable composite depots, which formed a non-flowing hydrogel, when the silica microparticles were mixed in 1:1 ratio (weight-to-volume) with the R400 silica sol, prepared as described above. With trisodium citrate the injections were easy with a 25 G needle (inner diameter 0.25-0.26 mm), but without citrate the injection was difficult, and the needle clogged.

### Example 2—Preparation of Silica Microparticles with and Without Active Pharmaceutical Ingredient and with and Without Pharmaceutically Non-Active Additives to Improve Rheological Properties and Injectability of Injectable Composite Depots

Example 2 illustrates how pharmaceutically non-active additive (trisodium citrate) can be used to adjust the properties of the silica microparticles and the injectable composite depots.

Silica microparticles without any encapsulated agents were prepared by spray-drying of a silica sol. Tetraethyl orthosilicate (TEOS), 0.1 M HCl, and deionized water were used in the preparation of the silica sol at pH 2 under strong mixing at room temperature. After the hydrolysis of TEOS, the resulting silica sol was cooled down to ca. 0° C. in an ice bath. Next, ethanol was added into the silica sol and 0.1 M NaOH was used for adjustment of pH to pH 5.5 prior to spray-drying resulting in molar H₂O: TEOS: HCl: EtOH ratio of 5:1:0.86:13.73.

Silica microparticles with an encapsulated active pharmaceutical ingredient (levothyroxine), as well as silica microparticles with an encapsulated active pharmaceutical ingredient (levothyroxine) and a pharmaceutically non-active additive were prepared by spray-drying of a silica sol. Trisodium citrate dihydrate was used as the pharmaceutically non-active additive for improving wetting, gel formation and injectability. The same silica sol was used both for the silica microparticles comprising levothyroxine, and for the silica microparticles comprising both levothyroxine and trisodium citrate dihydrate. The silica sol was prepared by hydrolysing TEOS in deionized water using 0.1 M HCl as a catalyst, the molar H₂O: TEOS: HCl ratio being 5:1:0.86. Levothyroxine sodium was dissolved in ethanol, and the solution was added into the silica sol, and pH was adjusted to pH 5.5 by adding 0.1 M NaOH. It resulted in the final H2O:TEOS:HCl:EtOH ratio of 5:1:0.86:28.98. For the silica microparticles comprising pharmaceutically non-active additive, trisodium citrate dihydrate was added into the silica sol-ethanol mixture when levothyroxine was fully dissolved. The final levothyroxine sodium concentration in the solution was 1.8 mg/ml, and trisodium citrate concentration was 0.6 mg/ml.

To prepare the microparticles, the silica sols 1) without active pharmaceutical ingredient and pharmaceutically non-active additive, 2) comprising levothyroxine as API but without pharmaceutically non-active additive, and 3) comprising both levothyroxine as API and trisodium citrate as pharmaceutically non-active additive, were all spray-dried with the same parameters. The silica sols were pumped under mixing into the spray-dryer Buechi B-290 (Büchi AG) at the flow rate of 5.6 ml/min. The aspirator air flow rate was 100%, atomization air flow was 670 l/h, total feed rate was 5.6 ml/min, and the inlet and outlet temperatures were 100° C. and 60° C.-70 ° C., respectively. The payload (in relation to the silica amount) of levothyroxine and trisodium citrate in the silica microparticles (when used) was 2 weight-% and 0.5 weight-%, respectively.

Thus three different silica microparticles were obtained:
1) without active pharmaceutical ingredient and pharmaceutically non-active additive,
2) comprising levothyroxine as API but without pharmaceutically non-active additive, and
3) comprising both levothyroxine as API and trisodium citrate as pharmaceutically non-active additive

The particle size distribution of the silica microparticles was measured with laser diffraction using HELOS 2370 (Sympatec). The particle size distribution is expressed with D10, D50 and D90, which indicate the size below which 10%, 50% or 90% of all particles are found. For the silica microparticles without API and pharmaceutically non-active additive, the particle size distribution was measured to be D10=1.09±0.00 μm, D50=3.02±0.00 μm, and D90=7.25±0.01 μm. For the silica microparticles comprising levothyroxine but no non-pharmaceutically active additive the particle size distribution was measured to be D10=1.15±0.02 μm, D50=4.17±0.01 μm, and D90=11.87 +0.04 μm. For the silica microparticles comprising both levothyroxine and trisodium citrate the particle size distribution was measured to be D10=1.09±0.00 μm, D50=3.02±0.00 μm and D90=7.25±0.01 μm. Hence, there is some difference in D90 value for the silica microparticles comprising levothyroxine as API, depending if a pharmaceutically non-active additive (trisodium citrate) is present in the silica microparticles or not. However, the difference has not been found to be significant from the viewpoint of injectability. In other words, the slight narrowing of the particle size distribution may have some effect, but the greater effect to the injectability comes from the differences in the surface chemistry, which have been observed in wetting difficulties and in prolonged gel formation time for the injectable composite depot, when not using a pharmaceutically non-active additive.

The silica microparticles were further used to prepare different injectable composite depots. One of the prepared injectable composite depots (0.5 g of silica microparticles in 1 ml in the R400 silica sol) comprises only silica as the solid phase (the all-silica depot), and it is used as a reference material in characterization of rheological properties and injectability of the different composite depots.

The preparation of different injectable composite depots using the three different silica microparticles (described above) started by preparation of a R400 silica sol (R400 meaning water and TEOS in molar ratio of 400:1, and the resulting silica content in the sol is ca. 0.82 weight-%). 0.1 M HCl was used as a catalyst by adjusting the pH to pH 2, and the precursors were mixed until TEOS was hydrolysed.

To prepare the all-silica depot (reference), the spray-dried silica microparticles without any encapsulated agents or additives were added into the R400 silica sol under mixing. The system thus comprised only silica as the solid phase. The resulting suspension, comprising silica microparticles in the R400 silica sol in 0.5:1 ratio (weight-to-volume), pH adjusted to pH 5.9 with 0.1 M NaOH, was transferred into syringes (1-ml BD Luer-Lok syringes, Becton, Dickinson and Company). The syringes were then kept under mixing in a custom-made rotating mixer (DelSiTech, Turku, Finland) at room temperature for 72 hours to ensure that a stable and non-flowing semisolid gel structure was formed. The formed all-silica composite was easily injectable through a 25 G needle (inner diameter 0.25-0.26 mm). The nanoparticles in the R400 silica sol merged onto the surface of the silica microparticles, which ensured that the suspension turned into a non-flowing and stable semi-solid hydrogel structure at rest. The semi-solid silica-silica hydrogel was also strongly shear-thinning, and thus easily injectable through thin needles, and it turned back into a non-flowing form after shear, e.g., after injection.

Preparation of a composite depot comprising silica microparticles with encapsulated levothyroxine, started by addition of the spray-dried microparticles into the silica sol (R400) under mixing. The resulting suspension, comprising silica microparticles in silica sol in 0.5-1.0: 1 ratio (weight-to-volume), pH adjusted to pH 5.9 with 0.1 M NaOH, was transferred into syringes (1-ml BD Luer-Lok syringes, Becton, Dickinson and Company). The syringes were then kept under mixing in a custom-made rotating mixer (DelSiTech, Turku, Finland) at room temperature for 72 hours. However, no non-flowing semisolid gel structure was formed, and when the material was injected through the 25G needle (inner diameter 0.25-0.26 mm), the needle was clogged, and the applied shear enhanced the phase-separation of the composite depot.

Next, an injectable composite depot comprising silica microparticles with encapsulated levothyroxine (2 weight-% in relation to the silica amount) and pharmaceutically non-active agent (trisodium citrate, 0.5 weight-% in relation to the silica amount) was prepared. The preparation started by adding of the spray-dried silica microparticles into the silica sol (R400) under mixing. The resulting suspension, comprising silica microparticles in the silica sol in 0.5:1 ratio (weight-to-volume), pH adjusted to pH 5.9 with 0.1 M NaOH, was transferred into syringes (1-ml BD Luer-Lok syringes, Becton, Dickinson and Company). The syringes were then kept under mixing in a custom-made rotating mixer (DelSiTech, Turku, Finland) at room temperature for 72 hours. A non-flowing and homogeneous gel structure was formed, and the composite depot was easily injectable through 25 G (inner diameter 0.25-0.26 mm) needle.

### Example 3—Improving Properties of Injectable Composite Depots by Pharmaceutically Non-Active Additives also in Diluted Silica Sol

Example 3 shows how the total silica amount, mainly coming from the silica microparticles, in an injectable composite depot can be decreased with help of a pharmaceutically non-active additive (alginate) in the silica sol. The pharmaceutically non-active additive in the silica sol also affects the rheological properties of injectable composite depots. The silica sol (R400) is not used for spray-drying, but for making the final injectable composite depot, where the silica nanoparticles of silica sol support the gel formation by merging to the surface of the silica microparticles. The decrease of total silica amount in the injectable composite depot can be utilized in adjustment of release rates of active pharmaceutical ingredients in composite depots, where the dissolution product of silica may exceed the sink conditions (free dissolution), or even reach saturated condition with respect to the silica dissolution products, e.g., in certain local sites in vivo. Biodegradable depots, which degrade by dissolution in the body fluids, e.g., in a vitreous after intravitreal injections, may reach a near-saturation level or saturation level due to limited space and a slow exchange rate of body fluids. The same may occur also in a subcutaneous, intramuscular, subconjunctival etc. space where the concentration of the dissolution products may be above the sink level (free dissolution). Hence, lower amount of silica in an injectable composite depot may also be used to fine-adjust the in vivo release rate of active pharmaceutical ingredients, e.g., as they are encapsulated in the silica microparticles, which is the major component of an injectable composite depot. The use of pharmaceutically non-active additives ensures proper rheological properties for the injectable composite depot.

Injectable composite depots comprising different amounts of spray-dried silica microparticles with and without encapsulated agent and pharmaceutically non-active second additive, as described in Example 2, were combined with a silica sol comprising a pharmaceutically non-active first additive (sodium alginate). The preparation of the injectable composite depots started with the preparation of the R400 silica sol, and after hydrolysis of tetraethyl orthosilicate (TEOS) at pH 2, the pH of the silica sol was adjusted to pH 3, after which sodium alginate was added under strong mixing at room temperature.

Different sodium alginate concentrations were used with different concentrations of the spray-dried silica microparticles from Example 2. The tested concentrations of sodium alginate in the silica sol (R400) were 0.25 weight-%, 0.50 weight-%, 0.75 weight-%, 1.00 weight-%, 1.25 weight-%, and 1.50 weight-%, calculated from the total weight of the silica sol and sodium alginate. The concentrations of the spray-dried silica microparticles with or without encapsulated agents and pharmaceutically non-active second additive ranged from 0.1 g to 1.0 g in 1 ml of the silica sol-alginate mixture (in 0.1:1-1:1 ratios, weight-to-volume). When the spray-dried silica microparticles were added into the silica sol-alginate mixture, the pH of the resulting suspension was adjusted to pH 5.9 using 0.1 M NaOH. Next, the suspension was transferred into syringes (1-ml BD Luer-Lok syringes, Becton, Dickinson and Company). The syringes were then kept under mixing in a custom-made rotating mixer at room temperature for 72 hours. Stable, non-flowing (at rest), semisolid, and easily injectable gel structures were then formed in most cases, but in the case of levothyroxine only in the silica microparticles, the material was more difficult to inject.

The spray-dried silica microparticles with encapsulated levothyroxine as API (2 weight-% in relation to the silica amount) and trisodium citrate as the pharmaceutically non-active second additive (0.5 weight-% in relation to the silica amount), described in Example 2, were used in preparation of the injectable composite depots with different concentrations of the pharmaceutically non-active first additive (sodium alginate) in the silica sol (R400). Different silica microparticle concentrations (0.1 g and 0.5 g in 1 ml of the silica sol with additive) were also tested. In the present example, 1 weight-% of alginate in the silica sol-alginate mixture was found to be advantageous with respect to the gel formation and injectability through a 25 G needle (inner diameter 0.25-0.26 mm) for the silica microparticle concentrations of 0.1-0.5 g silica microparticles in 1 ml of the silica sol-alginate mixture (0.1-0.5:1 ratio, weight-to-volume). It corresponds to 0.9 weight-%, in relation to the total mass of the composite depot, of pharmaceutically non-active additive (alginate), when 0.1 g of the silica microparticles were added into 1 ml of the silica sol-alginate mixture, and 0.7 weight-%, in relation to the total mass of the composite depot, of pharmaceutically non-active additive (alginate), when 0.5 g of the silica microparticles were added into 1 ml of the silica sol-alginate mixture. Although 1 weight-% of alginate in the silica sol was observed to be advantageous for 0.1-0.5 g microparticles in 1 ml of the silica sol-alginate mixture, also 0.75 weight-% and 1.25 weight-% of alginate were found effective with respect to the gel formation, rheology and injectability through a 25 G needle. For weight-to-volume ratios of 0.6-1.0 g of the silica microparticles in 1 ml of the silica sol-alginate mixture, 0.5-0.7 weight-% of alginate in the silica sol-alginate mixture was found to be advantageous. In case 0.75-1.0 weight-% of alginate was used, the result was stiff gels, which were difficult to inject.

Also the silica microparticles with encapsulated levothyroxine only (2 weight-% in relation to the silica amount) were tested for 0.1-0.5:1 ratio weight-to-volume in the silica sol comprising alginate as pharmaceutically non-active first additive. 1 weight-% of alginate was mixed in the silica sol, but the wetting was not good, and they did not form a non-flowing gel, and they could not be injected through a 25 G needle (inner diameter 0.25-0.26 mm).

Four different silica microparticles without encapsulated agents or pharmaceutically non-active additives were also tested. They were prepared from the silica sols R3-50, R5-50, R7.5-50 and R10-50, where the hydrolysis of tetraethyl orthosilicate (TEOS) at pH 2 was first done with molar water-to-TEOS ratio of 3, 5, 7.5, and 10, and after the hydrolysis of TEOS, the silica sols were diluted by adding ethanol in the volume that corresponds to the water volume to reach molar water-to-TEOS ratio of 50. The diluted silica sols were pumped under mixing into the spray-dryer Buechi B-290 (Buchi AG) at the flow rate of 5.6 ml/min. The aspirator air flow rate was 100%, atomization air flow was 670 l/h, total feed rate was 5.6 ml/min, and the inlet and outlet temperatures were 100° C. and 60° C.-70° C., respectively. The silica microparticles were then added in 0.1:1 weight-to-volume ratio into the silica sol (R400) mixed with alginate, comprising 0.5, 0.75 or 1.0 weight-% alginate. When the spray-dried silica microparticles were added into the silica sol comprising alginate, the pH of the resulting suspension was adjusted to pH 5.9 using 0.1 M NaOH. Next, the suspension was transferred into syringes (1-ml BD Luer-Lok syringes, Becton, Dickinson and Company). The syringes were then kept under mixing in a custom-made rotating mixer at room temperature for 72 hours. All formulations formed a relatively good gel structure, except the one comprising R3-50 silica microparticles. R10-50 formed the best gel structure, when using the silica sol (R400) comprising 1.0 weight-% of alginate. The R3-50 was also the worst with respect to wetting in the silica sol (R400) without any additives, and R10-50 was the best (fastest) in wetting, but none of them formed a gel in 0.1:1 weight-to-volume ratio without alginate. When using enough with alginate, an injectable composite depot was formed with R10-50. R5-50 worked well both with silica sol and with silica sol comprising alginate. This illustrates that there are also differences in the surface properties in the different silica microparticles without any additives, but with an appropriate amount of alginate in low microparticle concentrations a non-flowing gel structure can be formed, which also is injectable under shear when using thin needles. This information may be useful in the optimisation of injectable composite depots, because in some cases also use of silica microparticles without any additives can be used to fine-tune the rheological properties.

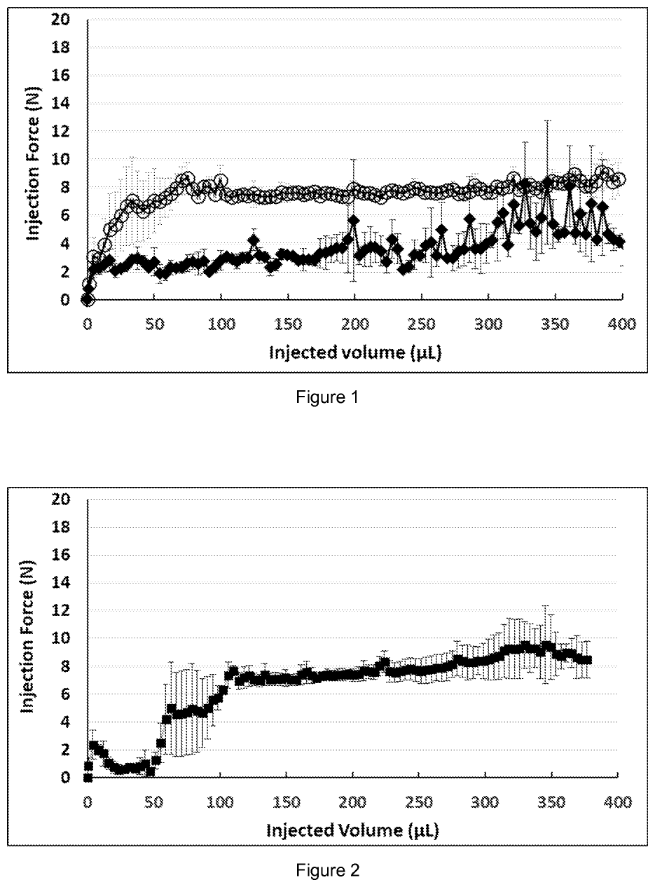

To illustrate the injectability of the composite depots, injection force measurements were conducted using a 25 G 1″ (needle gauge 0.5×25 mm) hypodermic needles (Fine-Ject, Henke Sass Wolf), which were attached to the prefilled syringes. The force required for injection for a single steady continuous push of the plunger was measured with a compressor equipment LR30K plus (Lloyd Instruments) with the 250 Newton load cell (grade 0.5%) at crosshead speed 1 mm/sec (60 mm/min). The injected volume was 0.3 ml. The results are shown in FIGS. 1-3.

FIG. 1 illustrates injection force needed for the injection through a 25 G needle (inner diameter 0.25-0.26 mm) for the injectable all-silica depot (filled diamonds) comprising spray-dried R5-50 silica microparticles without any encapsulated agents in 0.5:1 ratio weight-to-volume in the R400 silica sol (the all-silica depot), and in R400 silica sol comprising 1 weight-% alginate, calculated from total weight of the silica sol and alginate (open circles). FIG. 1 thus shows the injection force for 1) the reference depot (filled diamonds) comprising the spray-dried R5-50 silica microparticles without any encapsulated agents in 0.5:1 ratio weight-to-volume in the R400 silica sol only (the all-silica depot), and 2) for the inventive composite depot (open circles) comprising the spray-dried R5-50 silica microparticles without any encapsulated agents in 0.5:1 ratio weight-to-volume in R400 silica sol-alginate mixture (comprising 0.7 weight-% alginate in relation to the whole depot, i.e., 1% alginate in the R400-alginate mixture). It can be seen from FIG. 1 that all measured forces are below 20 N, and mostly below 10 N, meaning that they are commonly acceptable values for easy injection. With 0.5:1 ratio weight-to-volume, the forces are bit higher when the composite depot comprises alginate as pharmaceutically non-active additive, but the injection seems to be smoother than for the corresponding reference all-silica depot, i.e., there is more fluctuation in the reference all-silica depot due to larger microparticle aggregates or due to partial phase separation, which may make the injection a bit more difficult, although the material was easily injectable also in manual tests through a 25 G needle. The 0.5:1.0 ratio weight-to-volume is also close to the minimum for the reference all-silica depot (i.e., without any additives in the silica sol) with respect to the gel formation and good injectability, as the reference all-silica depot commonly better with weight-to-volume ratios larger than 0.5:1.0 up to 1.0:1.0.

FIG. 2 illustrates injection force needed for the injection through a 25 G needle (inner diameter 0.25-0.26 mm) for the injectable composite depot comprising silica microparticles with encapsulated levothyroxine (2 weight-% in relation to the silica amount) and trisodium citrate (0.5 weight-% in relation to the silica amount) in 0.5:1 ratio weight-to-volume in the R400 silica sol comprising 1 weight-% alginate, calculated from total weight of silica sol and alginate, i.e. silica sol comprising 0.7 weight-% alginate in relation to the whole composite depot. It can be seen from FIG. 2 that the trisodium citrate as the pharmaceutically non-active additive, present in the silica microparticles, improved the properties of the injectable composite depots and provided stable easily injectable depots. The silica microparticles with encapsulated levothyroxine and without pharmaceutically non-active additives did not form a gel structure at all, and hence also the injectability was bad (results not shown in FIG. 2). The silica microparticles with encapsulated levothyroxine, when the silica sol comprised 1 weight-% alginate as pharmaceutically non-active additive, calculated from silica sol-alginate mixture, were properly injectable through a 25 G needle (results not shown in FIG. 2). Thus, in the case of levothyroxine, the presence of trisodium citrate in the silica microparticles is preferable, and alginate in the silica sol makes the injection smoother in comparison to the reference all-silica depot.

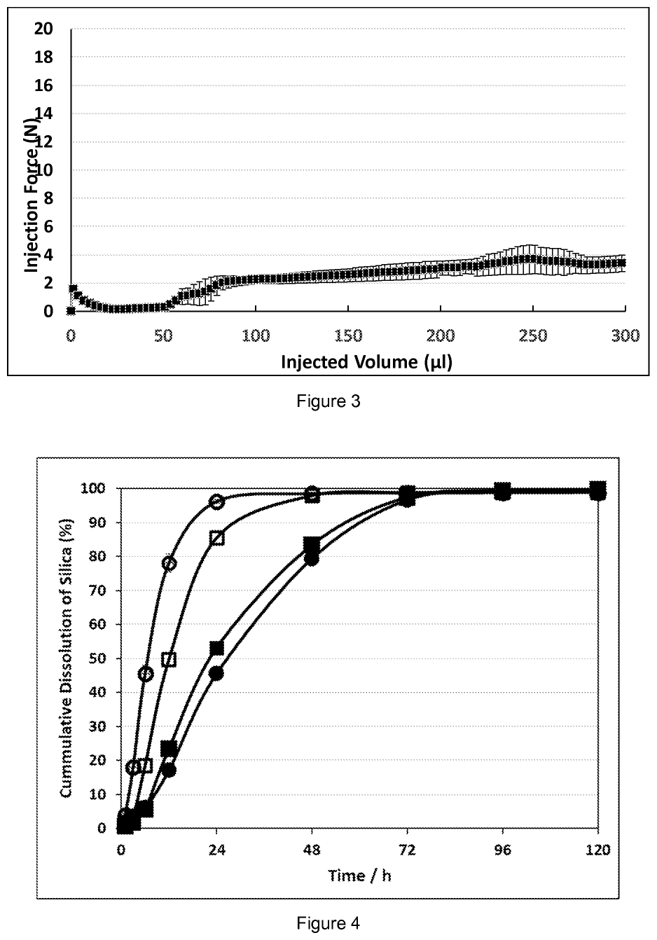

FIG. 3 illustrates injection force needed for the injection through a 25 G needle (inner diameter 0.25-0.26 mm) for the injectable composite depot comprising R5-100 silica microparticles with encapsulated levothyroxine (2 weight-% in relation to the silica amount) and trisodium citrate (0.5 weight-% in relation to the silica amount) in 0.1:1 ratio weight-to-volume in the R400 silica sol comprising 1 weight-% alginate, calculated from total weight of silica sol and alginate. It is seen in FIG. 3 that the injectable depot is easily injectable.

The injectable composite depots were further characterized by rheological measurements by Anton Paar rheometer (MCR302) using a parallel plate (d=25 mm) measuring geometry with the gap of 1.0 mm at 25° C. Both oscillatory measurements for elastic and viscous modulus and rotational measurements for shear-thinning viscosity were conducted. The oscillatory measurements were conducted in the linear viscoelastic region both before and after the rotational measurements to show that the injectable composite depot is a non-flowing gel structure both before and after the applied shear stress in the rotational measurement. The oscillatory measurements in the linear viscoelastic region simulate properties at rest with a minimal shear, wherein shear strain for all the studied materials was at 0.1%, and frequency at 1 Hz for 120 s before the shearing in the rotational measurements and 300 s after the shearing in the rotational measurements. Shear rate in the rotational measurements was kept constant at 1000 1/s for 30 s for all the studied depots.

The rheological results show that all the studied versions of the composite depots
1) the all-silica reference depot with 0.5:1 ratio weight-to-volume in the R400 silica sol (used in FIG. 1);
2) the depot with silica microparticles without any encapsulated agents in 0.5:1 ratio weight-to-volume in R400 silica sol comprising 1 weight-% alginate, calculated from total weight of the silica sol and alginate (used in FIG. 1); and
3) the injectable composite depots comprising levothyroxine and trisodium citrate with the 0.5:1 and 0.1:1 weight-to-volume ratios in the R400 silica sol comprising alginate (used in FIGS. 2 and 3);
are gels, non-flowing at rest, again after the high shearing in the viscosity measurements. The loss factors (the ratio between loss (viscous) modulus, G′ and storage (elastic) modulus), G′) were all below 1, which indicates that the materials are non-flowing as the storage (elastic) modulus is larger than the loss (viscous) modulus. The loss factor for the all-silica reference depot with 0.5:1 ratio weight-to-volume in R400 silica sol was 0.08-0.09 before shearing, and 0.147-0.07 after the shearing. The loss factor for the silica microparticles without encapsulated agents in 0.5:1 ratio weight-to-volume in the R400 silica sol comprising alginate was 0.15-0.16 before shearing, and 0.6-0.7 after the shearing. For the injectable composite depot comprising levothyroxine and trisodium citrate in the silica microparticles in 0.5:1 ratio weight-to-volume in the R400 silica sol comprising alginate, the loss factor was 0.007-0.01 before shearing, and 0.007-0.06 after the shearing. For the injectable composite depot comprising levothyroxine and trisodium citrate in the silica microparticles in 0.1:1 ratio weight-to-volume in the R400 silica sol comprising alginate, the loss factor was 0.15-0.16 before shearing, and 0.45-0.6 after the shearing. As expected, the values are lower for the injectable composite depots with 0.1:1 ratio weight-to-volume, but they are all non-flowing gels both before and after shearing.

The measurements for dissolution rate of silica and release rate of levothyroxine were conducted in 50 mM TRIS buffer pH 7.4 at 37° C. The dissolution conditions were kept in sink conditions (below ca. 20% of the solubility) both for silica and levothyroxine. 10-30 mg of silica microparticles and injectable composite depot samples were added in 50 ml of dissolution buffer, which was refreshed at every sampling time point to keep both the dissolved silica and levothyroxine in sink conditions. The dissolution studies were conducted for 5-7 days in a shaking water bath (Julabo Gmbh), 60 strokes/min, at 37° C. The dissolved silica concentration was measured with a Microwave plasma atomic emission spectrophotometer (MP-AES) (Agilent Technologies, 4210MP-AES) at wavelength of 251.611 nm. The released levothyroxine was quantified by HPLC (Agilent Technologies 1260 Infinity) with Phenomenex Luna 3 μm C18 (2), 150×4.0 mm. The mobile A phase consisted of 0.1% trifluoroacetic acid in water (v/v) and the mobile phase B consisted of 0.1% trifluoroacetic acid in acetonitrile (v/v). The absorbance was detected at 225 nm, the injection volume was 50 μl, the flow rate was 1.0 ml/min, and the column temperature was 25° C. The dissolution rate of silica and release rate of levothyroxine for the silica microparticles and for the injectable depots are shown in FIGS. 4-6.

FIG. 4 illustrates cumulative in vitro dissolution rate of silica in sink condition for the silica microparticles (R5-50) and different injectable composite depots comprising the R5-50 silica microparticles. FIG. 4 shows results for
a) silica microparticles, reference (open circles);
b) all-silica injectable reference depot comprising 0.5 g silica microparticles combined with silica sol (open squares);
c) injectable composite depot comprising 0.5 g silica microparticles combined with silica sol comprising alginate (filled squares); and
d) injectable composite depot comprising 0.1 g silica microparticles combined with silica sol comprising alginate (filled circles).

It is seen that for the all-silica injectable depot with the silica microparticles in R400 silica sol, the dissolution rate is a bit slower than that of the silica microparticles. The difference is a bit greater for the injectable depots comprising alginate and the same silica microparticles, but there is no practical difference for the different amount of the silica microparticles (0.1:1 vs. and 0.5:1 ratio) in the depots.

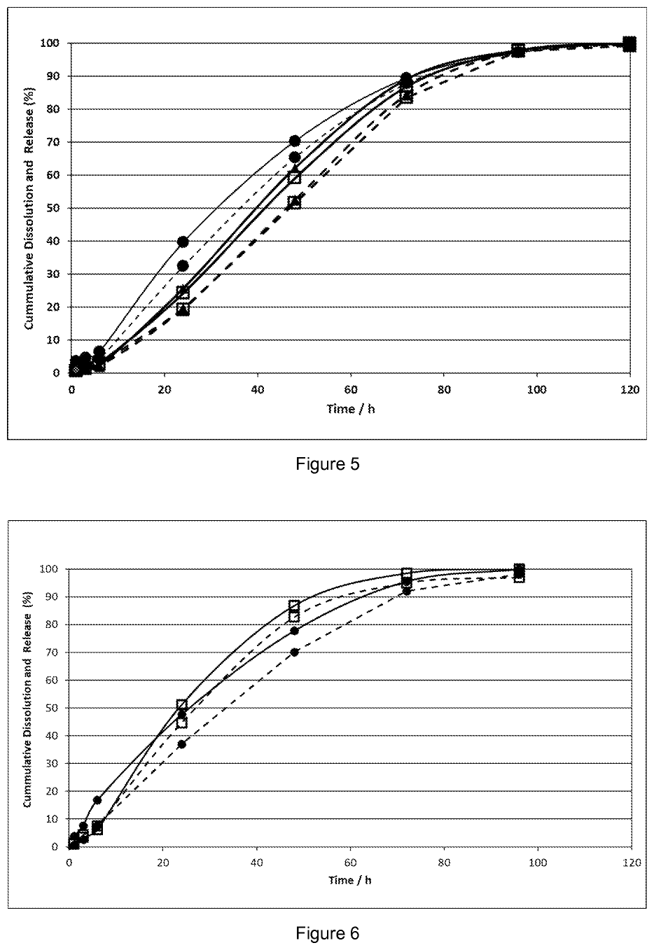

FIG. 5 illustrates cumulative in vitro dissolution rate of silica and release rate of levothyroxine. FIG. 5 shows dissolution results from
a) R5-50 silica microparticles comprising 2 weight-% of levothyroxine and 0.5 weight-% of trisodium citrate, in relation to silica amount in the microparticles. Results for levothyroxine shown as filled circles joined with solid line, for silica as filled circles joined with dash line;
b) injectable all-silica reference depot comprising 0.5 g silica microparticles comprising 2 weight-% of levothyroxine and 0.5 weight-% of trisodium citrate, in relation to silica amount in the microparticles, combined with 1 ml of R400 silica sol. Results for levothyroxine shown as filled triangles joined with solid line, for silica as filled triangles joined with dash line; and
c) injectable composite depot comprising 0.5 g microparticles comprising 2 weight-% of levothyroxine and 0.5 weight-% of trisodium citrate, in relation to silica amount in the microparticles, combined with 1 ml of R400 silica sol comprising 1 weight-% alginate, calculated from total with of silica sol and alginate. Results for levothyroxine shown as open squares joined with solid line, for silica as open squares joined with dash line.

FIG. 6 illustrates cumulative in vitro dissolution rate of silica and release rate of levothyroxine. FIG. 6 shows results from
a) R5-50 silica microparticles comprising 2 weight-% of levothyroxine and 0.5 weight-% of trisodium citrate, in relation to silica amount in the microparticles. Results for levothyroxine shown as filled circles joined with solid line, for silica as filled circles joined with dash line; and
b) injectable composite depot comprising 0.1 g microparticles comprising 2 weight-% of levothyroxine and 0.5 weight-% of trisodium citrate in relation to silica amount in the microparticles, combined with 1 ml of R400 silica sol comprising 1 weight-% alginate, calculated from total with of silica sol and alginate. Results for levothyroxine shown as open squares joined with solid line, for silica as open squares joined with dash line.

It is seen from FIGS. 5 and 6 that the effect of the non-flowing gel structure on the dissolution of silica and release of levothyroxine is not fully clear for the injectable depots, where the silica microparticles comprise levothyroxine and trisodium citrate are used. Generally, it can be said that the non-flowing gel structure of the injectable composite depots described here has some effect on the dissolution and release rates, but it seems that the effect on the dissolution rate is more dependent on the specific formulation of the silica microparticles.

### Example 4—Pharmaceutically Non-Active Additive, Trehalose, Added into Silica Sols to be Mixed with Spray-Dried Silica Microparticles, and Effect of Trehalose on Rheological Properties of Resulting Injectable Composite Depots

Silica microparticles without any encapsulated agents were prepared by spray-drying, and these microparticles were mixed with a R400 silica sol (molar water-to-TEOS ratio is 400), and with R400 silica sols comprising 1 weight-%, 10 weight-% and 20 weight-% of trehalose dihydrate to study the effect of the pharmaceutically non-active additive on the rheological properties of the resulting injectable composite depots.

A silica sol for spray-drying of the silica microparticles without any encapsulated agents was prepared by hydrolyzing tetraethyl orthosilicate (TEOS) in water, of which pH was adjusted to pH 2 using 0.1 M HCl. The molar water-to-TEOS ratio was 3. After the hydrolysis the sol was cooled down to ca. 0° C. in an ice-bath. Next, the molar water-to-TEOS ratio was raised from 3 to 50 (R3-50) by adding water and the pH was adjusted to ca. 5.0 by using 0.1 M NaOH. The R3-50 silica sol was then pumped into Buechi S300-1 spray-dryer with the flow rate of 6 ml/min, the inlet temperature was 100° C., the outlet temperature was 60-62° C., aspirator was set to 32 m³/h, and the atomization air flow was at 660-680 l/h.

The injectable composite depots were prepared by mixing the R3-50 silica microparticles with the R400 silica sol and with the R400 silica sols comprising 1 weight-%, 10 weight-% and 20 weight-% of trehalose. Before adding trehalose into the R400 silica sol, the pH was first raised to pH4.5 and then the silica microparticles were added (0.5 g in 1 ml of R400 silica sols with and without added trehalose) under mixing, and the pH was further raised to pH6. The suspensions were transferred into syringes and the syringes were placed into a roller mixer for 72 hours to ensure the formation of the injectable composite depot structure, a hydrogel, which is non-flowing at rest.

The resulting injectable composite depots were further characterized by rheological measurements by Anton Paar rheometer (MCR302) using a parallel serrated plate (d=10 mm) measuring geometry with the gap of 1.0 mm at 25° C. Oscillatory measurements for elastic (storage) and viscous (loss) modulus were conducted. The oscillatory measurements were conducted in the linear viscoelastic region, which simulates the behaviour of the materials at rest.

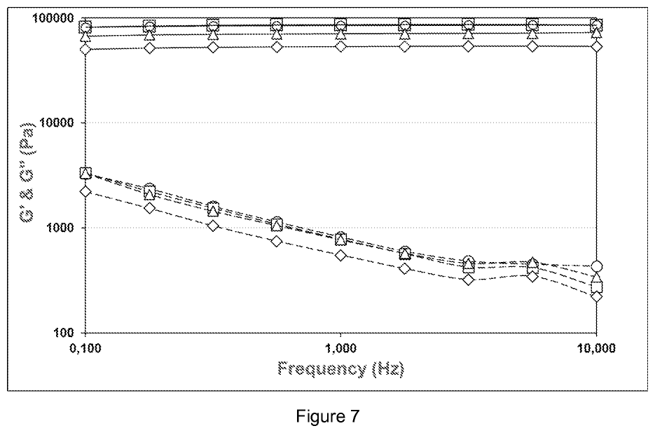

FIG. 7 illustrates the viscoelastic properties, i.e. storage (elastic) and loss (viscous) modulus, for the injectable composite depots comprising silica microparticles, which are mixed with the R400 silica sol with and without a pharmaceutically non-active additive, trehalose. FIG. 7 shows results for
a) Storage modulus (G′) for the injectable composite depot comprising the silica microparticles in the R400 silica sol with no additive (open circles, smooth line);
b) Storage modulus (G′) for the injectable composite depot comprising the silica microparticles in the R400 silica sol which comprises 1 weight-% of trehalose (open rectangles, smooth line);
c) Storage modulus (G′) for the injectable composite depot comprising the silica microparticles in the R400 silica sol which comprise 10 weight-% of trehalose (open triangles, smooth line);
d) Storage modulus (G′) for the injectable composite depot comprising the silica microparticles in the R400 silica sol which comprise 20 weight-% of trehalose (open diamonds, smooth line);
e) Loss modulus (G′) for the injectable composite depot comprising the silica microparticles in the R400 silica with no additive (open circles, dashed line);
f) Loss modulus (G′) for the injectable composite depot comprising the silica microparticles in the R400 silica sol which comprise 1 weight-% of trehalose (open rectangles, dashed line);
g) Loss modulus (G′) for the injectable composite depot comprising the silica microparticles in the R400 silica sol which comprise 10 weight-% of trehalose (open triangles, dashed line);
h) Loss modulus (G′) for the injectable composite depot comprising the silica microparticles in the R400 silica sol which comprise 20 weight-% of trehalose (open diamonds, dashed line).

As seen in FIG. 7, the storage and loss moduli do not differ much from each other, but there is a slight decreasing trend in the moduli as the trehalose concentration in the silica sol is increasing. The injectable composite depots have lower moduli values as the trehalose concentration in the R400 silica sol is increased, but they all are still proper non-flowing hydrogels at rest. The storage moduli are almost the same (ranging from 81 to 86 kPa at 0.1-10 Hz) for the injectable composite depot without any additive, and for the injectable composite depot comprising 1 weight-% of trehalose in the R400 silica sol. The storage moduli for the for the injectable composite depot comprising 10 weight-% and 20 weight-% of trehalose in the R400 silica sol, the storage moduli at 0.1-10 Hz varied between 67-73 kPa and 50-54 kPa, respectively. The loss factors (tan δ=G″/G′) of the injectable composite depots were very close to each other. At 0.1 Hz they were all within 0.04-0.05, at 1 Hz within 0.009-0.011, and at 10 Hz within 0.003-0.005, i.e., they were clearly below 1 meaning that all the studied injectable composite depots were clearly non-flowing hydrogels at rest. They are also easily injectable from syringes through thin needles, like 25 G needles.

The results shown in FIG. 7 are for the injectable composite depots prepared from a suspension comprising 0.5 g silica microparticles in 1 ml of R400 silica sol without any additive or 0.5 g silica microparticles in 1 ml R400 silica sol comprising either 1, 10 or 20 weight-% of trehalose. The results show that the pharmaceutically non-active additive, trehalose, can be used to adjust and fine-tune the rheological properties of the injectable composite depots. The good injectability of the injectable composite depots may depend on small changes in the rheology caused by active pharmaceutical ingredients. It has been observed that the active pharmaceutical ingredients often change the surface chemistry of the silica microparticles. The changes in surface chemistry may strongly depend on the active pharmaceutical ingredient and/or its payload. The presence of the pharmaceutically non-active additive affects the hydrogel formation mechanism providing a possibility to control the rheological properties of the injectable composite depot, but it does not prevent the hydrogel formation. The pharmaceutically non-active additive, e.g. trehalose, may be used to decrease the viscosity, if the surface chemistry of the silica microparticles comprising an active pharmaceutical ingredient has changed the rheology for example so that a thicker, and less patient-compliant needle should be used for parenteral injection of the injectable composite depot. Furthermore, especially the rheological properties of injectable composite depots comprising low concentrations of silica microparticles are more dependent on surface chemistry variations. Even in these cases, the pharmaceutically non-active additives, such as trehalose, can be used to adjust the formation of the non-flowing hydrogel structure of the injectable composite depots, and the rheological properties in general.

Even if the invention was described with reference to what at present seems to be the most practical and preferred embodiments, it is appreciated that the invention shall not be limited to the embodiments described above, but the invention is intended to cover also different modifications and equivalent technical solutions within the scope of the enclosed claims. Thus, the described embodiments are illustrative and should not be construed as restrictive.

1. An injectable composite depot, which is shear-thinning and comprises
a) up to 85 weight-%, preferably up to 80 weight-%, of silica microparticles having a maximum diameter of ≤1000 μm, and preferably comprising at least one encapsulated biologically active agent, combined with
b) a silica sol having a silica content of ≤5 weight-%, preferably ≤2 weight-%, more preferably ≤1 weight-%,
wherein the injectable composite depot comprises at least one pharmaceutically non-active additive encapsulated in the silica microparticles and/or present in the silica sol.

2. The injectable composite depot according to claim 1, characterized in that the at least one pharmaceutically non-active additive present in the silica sol comprises a first additive selected from monosaccharides, disaccharides, oligosaccharides, polysaccharides, or their salts, or any combinations thereof; preferably from oligosaccharides, polysaccharides, or their salts or any combinations thereof; more preferably from polysaccharides or their salts, even more preferably from alginate or its salts.

3. The injectable composite depot according to claim 2, characterized in that the first additive in the silica sol has a concentration in a range of 0.25-1.5 weight-%, preferably 0.50-1.25 weight-%, more preferably 0.75-1.25 weight-% or 0.75-1.0 weight-%, calculated from the total weight of the silica sol and the pharmaceutically non-active additive(s).

4. The injectable composite depot according to claim 1, characterized in that the at least one pharmaceutically non-active additive comprises a second additive selected from citric acid or salts of citric acid, preferably from monosodium citrate or its hydrates, disodium citrate or its hydrates, or trisodium citrate or its hydrates, and most preferably from trisodium citrate or its hydrates.

5. The injectable composite depot according to claim 4, characterized in that the second additive has a concentration ≤5 weight-%, preferably in range of 0.1-5 weight-%, preferably 0.1-3 weight-%, calculated from a total weight of the silica in the injectable composite depot.

6. The injectable composite depot according to claim 4, characterized in that the second additive is both encapsulated in the silica microparticles and present in the silica sol.

7. The injectable composite depot according to claim 1, characterized in that the pharmaceutically non-active additive(s) encapsulated in the silica microparticles and the pharmaceutically non-active additive(s) present in the silica sol are different from each other.

8. The injectable composite depot according to claim 1, characterized in that the silica microparticles comprise 0.01-70 weight-%, preferably 0.3-50 weight-%, more preferably 1-30 weight-%, of biologically active agents, calculated from the total weight of silica microparticle.

9. The injectable composite depot according to claim 1, characterized in that the silica microparticles have a diameter in a range of 1-300 μm, preferably 1-100 μm, more preferably 1-30 μm, even more preferably 0.5-20 μm.

10. The injectable composite depot according to claim 1, characterized in that the silica sol comprises silica nanoparticles having a diameter in a range of 10-1000 nm, preferably 10-500 nm, and more preferably 10-190 nm.

11. The injectable composite depot according to claim 1, characterized in that the injectable composite depot comprises 5-85 weight-%, preferably 5-80 weight-%, more preferably 5-50 weight-%, of the silica microparticles, calculated from the total weight of the injectable composite depot.

12. An injectable formulation comprising the injectable composite depot according to claim 1, preferably comprising a biologically active agent.

13. A method of use of the injectable composite depot of claim 1 for administering a biologically active agent or agents, preferably an active pharmaceutical ingredient or ingredients.

14. The method of use according to claim 13, characterized in that administration is topical or parenteral.

15. The method of use according to claim 14, characterized in that administration is topical and a topical biologically active agent is administered either as eye drops, creams, ointments, lotions, or suspensions.

16. The method of use according to claim 15, characterized in that the pharmaceutically non-active additive is trehalose.

17. The method of use according to claim 14 characterized in that administration is parenteral and selected from the group consisting of intravenous, intraarterial, intracardiac, transdermal, transmucosal, intradermal, subcutaneous, intramuscular, intraperitoneal, intracerebral, intracerebroventricular, intrathecal, intraosseous, intraarticular, ophthalmic, intraocular, intravitreal, subconjunctival, intracameral, subretinal, retrobulbar, peribulbar, suprachoroidal, periocular, transscleral, intrasternal, posterior juxtascleral, sub-tenon, intravesical and intracavernosal.

18. The injectable composite depot of claim 1 for use as medicament.
